(19) United States
(12) Patent Application Publication
(10) Pub. No.: US 2021/0341338 A1
(43) Pub. Date: Nov. 4, 2021

(54) Remote Aggregated Monitoring of Wellness

(71) Applicant: Augmented Radar Imaging, Inc., Los Altos, CA (US)

(72) Inventor: Victor Shtrom, Los altos, CA (US)

(73) Assignee: Augmented Radar Imaging, Inc., Los Altos, CA (US)

(21) Appl. No.: 16/866,457

(22) Filed: May 4, 2020

(51) Int. Cl. G01J 5/00; G06Q 50/26; G16H 40/67; G16H 50/20; G01S 13/34; G01S 13/86; A61B 5/01; A61B 5/00; A61B 5/08; A61B 5/11; G08B 21/02

(52) U.S. Cl. CPC G01J 5/0025; G06Q 50/265; G16H 40/67; G16H 50/20; G01S 13/34; G01S 13/862; G08B 21/02; A61B 5/015; A61B 5/746; A61B 5/0816; A61B 5/112; A61B 5/7264; G01S 13/865

(57) ABSTRACT

An electronic device that selectively performs a remedial action is described. During operation, a measurement sensor in the electronic device remotely performs measurements corresponding to temperatures of a group of individuals. Moreover, an integrated circuit in the electronic device may determine a parameter corresponding to a distribution of inferred temperatures of the group of individuals based at least in part on the measurements. Then, the integrated circuit may identify an individual in the group of individuals based at least in part on the determined parameter and an inferred temperature of the individual. Next, the electronic device may selectively perform the remedial action based at least in part on the identification of the individual.

### BACKGROUND

### Field

The described embodiments relate to monitoring of wellness. Notably, an electronic device remotely monitors a group of individuals and identifies wellness changes of individuals relative to the group.

### Related Art

A variety of public health initiatives are used to help address an outbreak of infectious disease. For example, public health surveillance may be used, on an ongoing basis, to collect, analyze and interpret data in an attempt to identify affected or symptomatic individuals. This data may be disseminated to, e.g., public health departments, which then use the information to try to prevent and control further spreading of disease and/or injury.

However, in this approach, the data collection is typically limited particular locations and types of organizations, and the decision making regarding any subsequent action is often centralized. For example, many public health departments require doctors and hospitals to report incidents of disease occurrence (such as patients that have particular symptoms) or diagnosis. While this information can be useful in guiding subsequent public health initiatives, the collected data may be biased or inaccurate. Notably, only a subset of the patients with a disease typically seek medical treatment, and there is often a lag between disease onset and when a given patient seeks medical help. Consequently, the data collected from medical professionals is filtered (it is a sub-sampling of the total patient population) and delayed, which can make it more difficult to make timely public-health decisions and to implement effective policies to manage the disease.

Alternatively, in some public health surveillance protocols, data is collected at key locations, such as at transportation hubs (e.g., airports or train stations). Notably, public health workers may setup checkpoints where individuals that are passing through are assessed, such as by taking their temperatures using a medical-grade forehead thermometer. While a forehead thermometer does not involve contact with an individual, it typically requires that a public-health worker, who is performing the measurement, be in close proximity with the individual being assessed, which increases the risk of disease transmission. Moreover, this type of monitoring is relatively time consuming, which can make a checkpoint into a bottleneck or choke point. The resulting lines of individuals waiting to be screened makes the checkpoints inconvenient and can also increase the risk of disease transmission. Furthermore, the use of medical-grade forehead thermometers and public-health workers increases the expense of this type of monitoring. Consequently, it can be difficult to scale distributed monitoring throughout a city or a region.

### SUMMARY

An electronic device that selectively performs a remedial action is described. This electronic device includes: a measurement sensor having a field of view that remotely performs measurements corresponding to temperatures of a group of individuals; and an integrated circuit (such as a processor), coupled to the measurement sensor, that analyzes the measurements. Notably, during operation, the measurement sensor remotely performs measurements corresponding to the temperatures of a group of individuals. Moreover, the integrated circuit may determine a parameter corresponding to a distribution of inferred temperatures of the group of individuals based at least in part on the measurements. Then, the integrated circuit may identify an individual in the group of individuals based at least in part on the determined parameter and an inferred temperature of the individual. Next, the electronic device may selectively perform the remedial action based at least in part on the identification of the individual.

For example, the measurement sensor may include an infrared sensor, such as a thermopile or a set of thermocouples. Moreover, the measurement sensor may have an accuracy that is greater than medical grade, such as an inferred temperature accuracy greater than 0.1 or 0.5 C. Consequently, the measurement sensor may provide qualitative or relative measurements of the temperatures.

Furthermore, the parameter may include or may be related to a moment of the distribution, such as an average, a mean and/or a non-zero number times a standard deviation of the distribution. Alternatively or additionally, the parameter may include a percentile or may correspond to a portion of the distribution, such as a 75^{th}, 90^{th} or 95^{th} percentile.

In some embodiments, the identified individual may be potentially unwell.

Moreover, the remedial action may include: providing an acoustic or a visual alert; providing a message; performing one or more additional measurements on the individual; identifying one or more other individuals that have been in proximity with the individual during a time interval; enforcing a social distance between the individual and a remainder of the group of individuals; or changing an access control state of a portal.

Furthermore, the determining of the parameter corresponding to the distribution of the inferred temperatures may calibrate the measurement sensor for a change in an environmental condition, such as a change in a temperature of an environment that includes the group of individuals and the measurement sensor.

Additionally, the remote measurements may be performed on the group of individuals at a distance greater than a fraction of an average height of the group of individuals.

In some embodiments, the electronic device may include one or more additional types of measurement sensors that remotely perform one or more additional types of measurements on the group of individuals. For example, the one or more additional types of measurements may include: acoustic measurements, optical imaging, LIDAR, radar measurements, and/or weight measurements. Note that the radar measurements may use a frequency-modulated continuous-wave (FMCW) radar signal, where a carrier frequency of the FMCW radar signal has a predefined variation as a function of time (such as: a sine wave, a sawtooth wave or a chirp, a triangle wave and/or a square wave) over a predefined frequency range. Moreover, note that the one or more additional types of measurements may be performed concurrently with the measurements. However, in other embodiments at least some of the measurements and the one or more additional types of measurements are performed sequentially.

Furthermore, the integrated circuit may analyze the one or more additional types of measurements to determine: a type of vital sign of the group of individuals, breathing styles of the group of individuals, walking speeds of the group of individuals, gait styles of the group of individuals, etc. Additionally, the individual may be identified based at least in part on the one or more additional types of measurements, such as the analysis of the one or more additional types of measurements. In some embodiments, the measurements and/or the one or more additional types of measurements are analyzed using a machine-learning model and/or a neural network. For example, the machine-learning model and/or the neural network may be pretrained or predetermined to identify the individual based at least in part on the measurements and/or the one or more additional types of measurements.

Another embodiment provides a computer-readable storage medium for use with the electronic device. This computer-readable storage medium may include program instructions that, when executed by the electronic device, causes the electronic device to perform at least some of the aforementioned operations of the electronic device.

Another embodiment provides a method for selectively performing a remedial action. The method includes at least some of the aforementioned operations performed by the electronic device.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a drawing illustrating an example of an environment that includes an electronic device in accordance with an embodiment of the present disclosure.

FIG. 2 is a flow diagram illustrating an example of a method for selectively performing a remedial action in accordance with an embodiment of the present disclosure.

FIG. 3 is a drawing illustrating an example of communication between components in an electronic device in FIG. 1 in accordance with an embodiment of the present disclosure.

FIG. 4 is a drawing illustrating an example of monitoring wellness using an electronic device of FIG. 1 in accordance with an embodiment of the present disclosure.

FIG. 5 is a block diagram illustrating an example of an electronic device in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

An electronic device that selectively performs a remedial action is described. During operation, a measurement sensor in the electronic device remotely performs measurements corresponding to temperatures of a group of individuals. Moreover, an integrated circuit in the electronic device may determine a parameter corresponding to a distribution of inferred temperatures of the group of individuals based at least in part on the measurements. Then, the integrated circuit may identify an individual in the group of individuals based at least in part on the determined parameter and an inferred temperature of the individual. Next, the electronic device may selectively perform the remedial action based at least in part on the identification of the individual.

By selectively performing the remedial action, these monitoring techniques may provide robust, remote monitoring of wellness of the group of individuals. For example, the monitoring techniques may be used at different, arbitrary locations, thereby providing distributed monitoring. Moreover, the monitoring techniques may be performed efficiently and in a non-invasive manner, and may be performed at a distance from the group of individuals. Consequently, the monitoring techniques may not be time consuming, may avoid crowding or having individuals in proximity of each other, so the monitoring techniques may not increase the risk of disease transmission. In addition, by using data for the group of individuals, the monitoring techniques may allow the use of a non-medical-grade measurement sensor, which may reduce the cost of the electronic device. Therefore, the monitoring techniques may provide low-cost, flexible monitoring of the wellness of the group of individuals, and may facilitate rapid performing of the remedial action when the individual is identified. In these ways, the monitoring techniques may promote public health, including: public-health surveillance and related public-health initiatives that manage disease and its spread.

In the discussion that follows, an infrared sensor is used as an illustrating of the measurement sensor. For example, the infrared sensor may be a passive infrared sensor, including: a bolometer (such as a thermopile or a set of thermocouples), a pyroelectric detector, or a photonic detector (such as a photoconductive detector or a photovoltaic detector) using a semiconductor having a narrow band gap (such as mercury cadmium telluride, a II-VI semiconductor alloy, etc.). Alternatively, the infrared sensor may be an active infrared sensor, which emit and detect infrared radiation.

However, a wide variety of measurement techniques may be used in the monitoring techniques, including: acoustic measurements (such as acoustic measurements using a microphone, a phase-array of microphones, etc.), optical imaging in the visible spectrum or a visible frequency band (such as images captured using an image sensor, e.g., a CMOS or a CCD image sensor), imaging in a different frequency band (such as sonar, acoustic, ultrasound, ultraviolet, x-ray, etc.), radar measurements, light detection and ranging (LIDAR), and/or weight measurements (such as weight measurements captured using a scale). Note that the radar signals may be continuous wave and/or pulsed, may modulated (such as using frequency modulation or pulse modulation) and/or may be polarized. For example, the radar signals may be pulse-modulated continuous-wave. Alternatively or additionally, the radar measurements may use a frequency-modulated continuous-wave (FMCW) radar signal, where a carrier frequency of the FMCW radar signal has a predefined variation as a function of time (such as: a sine wave, a sawtooth wave or a chirp, a triangle wave and/or a square wave) over a predefined frequency range (such as a carrier frequency that varies between 59 and 64 GHz over 10 μs). For example, the radar may involve radar signals having a fundamental or carrier frequency of 24 GHz, 59-64 GHz, 76-81 GHz or 140 GHz (which correspond to a fundamental or carrier wavelength of 0.01249 m, 5.08-4.68 mm, 3.95-3.701 mm or 2.14 mm), and/or another electromagnetic signal having a fundamental frequency in the radio or microwave frequency band.

Moreover, in the discussion that follows, the electronic device may optionally communicate using one or more of a wide variety of communication protocols. For example, the communication may involve wired and/or wireless communication. Consequently, the communication protocols may include: an Institute of Electrical and Electronics Engineers (IEEE) 802.11 standard (which is sometimes referred to as ‘Wi-Fi®,’ from the Wi-Fi Alliance of Austin, Tex.), Bluetooth® (from the Bluetooth Special Interest Group of Kirkland, Wash.), another type of wireless interface (such as another wireless-local-area-network interface), a cellular-telephone communication protocol (e.g., a 3G/4G/5G communication protocol, such as UMTS, LTE, etc.), an IEEE 802.3 standard (which is sometimes referred to as ‘Ethernet’), etc.

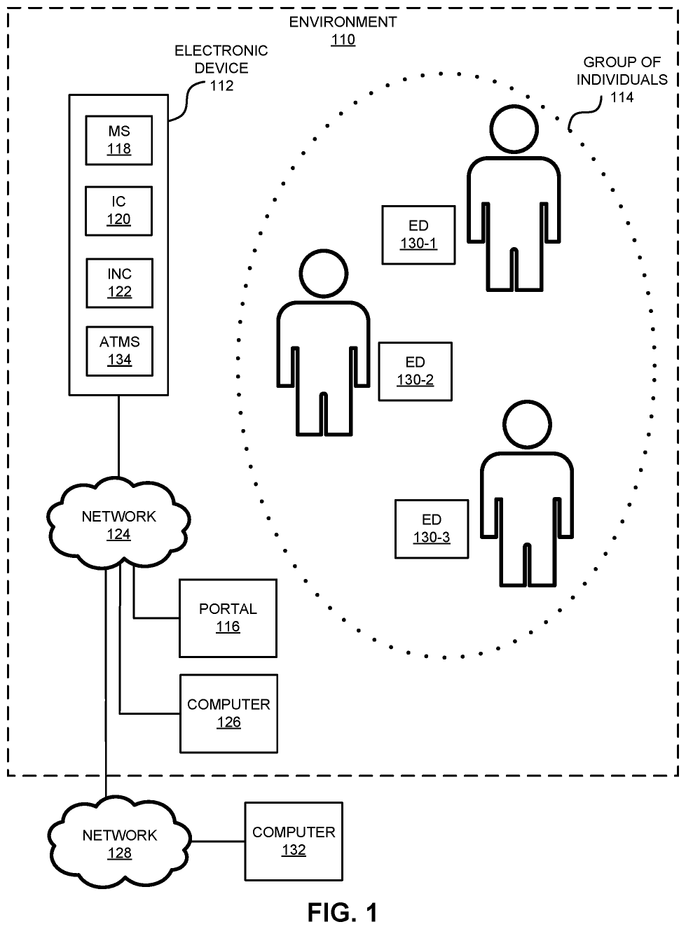

We now describe some embodiments of the monitoring techniques. FIG. 1 presents a drawing illustrating an example of an environment 110 that includes an electronic device 112. Moreover, a group of individuals 114 may be located in environment 110, such as: a building, an entrance lobby of the building, a room, an airport checkpoint, a train station, an indoor or an outdoor mall, a hallway or a walkway, a region where people gather, etc. For example, the group of individuals 114 may be walking or approaching a portal 116, such as a door, a security checkpoint, etc.

As discussed previously, in order to reduce or eliminate the spread of an infectious disease in environment 110, it may be useful to collect data about the group of individuals 114. However, performing the monitoring using one or more public-health workers, e.g., by having them take temperatures, is expensive and time-consuming, and may increase the risk of disease transmission.

In order to address these problems, electronic device 112 may perform, at least in part, operations in the monitoring techniques. Electronic device 112 may monitor wellness of the group of individuals 114 and, when an outlier or potentially unwell individual is identified, may selectively perform a remedial action. Moreover, electronic device 112 may include: a measurement sensor (MS) 118 having a field of view that remotely (and, thus, non-invasively) performs measurements corresponding to temperatures of the group of individuals 112; and an integrated circuit (IC) 120 (such as a processor), coupled to measurement sensor 118, that analyzes the measurements.

During operation, measurement sensor 118 may remotely perform measurements corresponding to the temperatures of the group of individuals 114. For example, measurement sensor 118 may include an infrared sensor. The measurements on individuals in the group of individuals 114 may be performed separately (such as sequentially as the individuals approach electronic device 112 and/or portal 116) or concurrently (such as when the group of individuals 114 are within the field of view of measurement sensor 118). Moreover, measurement sensor 118 may have an accuracy that is greater than medical grade, such as an inferred temperature accuracy based at least in part on the measurements that is greater than 0.1 or 0.5 C. Consequently, measurement sensor 118 may provide qualitative or relative measurements of the temperatures of the group of individuals 114. In some embodiments, measurement sensor 118 may include one or more Fresnel lenses that focus infrared electromagnetic signals and define the field of view of measurement sensor 118, such as 90°, 180°, 270° or 360°. Note that measurement sensor 118 may perform the measurements on the group of individuals 114 at a distance greater than a fraction of an average height of the group of individuals 114, such as a distance greater than 1-6 ft.

Moreover, integrated circuit 120 may determine a parameter corresponding to a distribution of inferred temperatures of the group of individuals 114 based at least in part on the measurements. For example, the parameter may include or may be related to a moment of the distribution, such as an average, a mean and/or a non-zero number times a standard deviation of the distribution. Alternatively or additionally, the parameter may include a percentile or may correspond to a portion of the distribution, such as a 75^{th}, 90^{th}, 95^{th} or 99^{th} percentile (e.g., the 75^{th}, 90^{th}, 95^{th} or 99^{th} largest inferred temperatures of the group of individuals 114). Note that using the percentile may also provide information about the confidence interval.

Then, integrated circuit 120 may identify an individual in the group of individuals 114 based at least in part on the determined parameter and an inferred temperature of the individual. For example, the individual may be identified as being potentially unwell when the inferred temperature of the individual is an outlier in the distribution of inferred temperatures, such as when the inferred temperature of the individual is more than 1.5, 2, or 3 standard deviations from a mean of the distribution of inferred temperatures or when the inferred temperature of the individual is above the percentile of the distribution of inferred temperatures.

Next, electronic device 112 may selectively perform the remedial action based at least in part on the identification of the individual. For example, an interface circuit (INC) 122 (such as a radio or a network interface) in electronic device 112 may provide, via network 124 (such as a wired and/or a wireless network, e.g., a wireless local area network or WLAN, and/or a cellular-telephone network), a message to a computer 126.

This message may include an alert, which may result in audio output (such as an alarm) or a visual indication (such as a flashing light).

Alternatively or additionally, the remedial action may include electronic device 112 performing one or more additional measurements on the individual using the same type of measurement (which may be performed using the measurement sensor 118 or another measurement sensor with a higher accuracy) or a different type of measurement. The one or more additional measurements may determine other characteristics of the individual that may improve the accuracy of the identification, such as that the individual is potentially unwell.

Moreover, the remedial action may include electronic device 112 identifying one or more other individuals that have been in proximity with the individual during a time interval, such as a preceding 1-3 days. For example, electronic device 112 may access, via network 124 and/or 128 (such as an intranet and/or the Internet), location information of the individual and the one or more other individuals during the time interval. This location information may include monitored locations (e.g., using a Global Positioning System, a WLAN, a cellular-telephone network, etc.) of other electronic devices (such as electronic devices 130, e.g., cellular telephones, associated with the individual and the one or more other individuals) and/or proximity information (which may be stored groups of two or more of the other electronic devices when they come within proximity of each other, such as 6-18 ft., or when they are in the same room or enclosed space). Alternatively or additionally, the location information may be determined by one or more camera systems (not shown) that capture images (e.g., in environment 110 and/or other locations) and then analyze them to identify the individual and the one or more other individuals. Note that electronic devices 118 and/or the one or more camera systems may provide, via network 124 and/or 128, the location information to electronic device 112, computer 126 and/or computer 132, e.g., using wireless and/or wired communication. Thus, when performing the remedial action, electronic device 112 may access the location information locally (e.g., stored in electronic device 112) and/or remotely (e.g., from computer 126 and/or computer 132).

In some embodiments, the identifying of the one or more other individuals may facilitate contact tracing and/or providing a notification to the one or more other individuals that the individual (or an anonymous individual with whom the one or more other individuals were in proximity during the time interval) was identified as being potentially unwell.

Furthermore, when the individual is identified, the remedial action may include electronic device 112 providing, via network 124, an instruction to portal 116 to change an access control state of a portal 116. For example, based at least in part on the instructions, portal 116 may be locked and may not open when the individual provides access credentials (such as when the individual swipes an access card through a reader or places the access card in proximity of the reader). This may deny the identified individual access or the ability to pass through portal 116.

Additionally, the remedial action may include electronic device 112 enforcing a social distance (such as 6 ft of separation) between the individual and a remainder of the group of individuals. For example, electronic device 112 may provide (via a speaker) a generic audio instruction regarding social distancing when the individual is identified, or may provide a specific audio instruction to maintain the social distance from the individual when the individual is identified. In some embodiments, electronic device 112 may provide, via network 124, a message to one of electronic devices 130 (such as electronic device 130-1), which is associated with the individual, to maintain the social distance from a remainder of the group of individuals 114.

Note that, in some embodiments, the remedial action may include electronic device 112 requesting, via network 124 and/or 128, police or medical assistance for the identified individual.

While the preceding discussion illustrated electronic device 112 performing operations in the monitoring techniques, in some embodiments at least some of these operations are performed by another electronic device. For example, in some embodiments, electronic device 112 may provide, via network 124 and/or 128, the measurements and/or the inferred temperatures to computer 124 and/or 132, and computer 124 and/or 132 may: determine the parameter, identify the individual and selectively perform the remedial action. Thus, in some embodiments, at least some of these operations in the monitoring techniques are performed remotely from electronic device 112 (such as by using a cloud-based computer, e.g., computer 132).

In some embodiments, the determining of the parameter corresponding to the distribution of the inferred temperatures may calibrate measurement sensor 118 for a change in an environmental condition in environment 110, such as a change in a temperature of environment 110 that includes the group of individuals 114 and measurement sensor 118 (such as a temperature change of environment 110 of more than 1 C). For example, in order to infer the temperatures of the group of individuals 114 from the measurements, integrated circuit 120 may not be able to use differential detection (such as a differential amplifier). Notably, in order to infer temperature from a measurement, many infrared temperature sensors evaluate or analyze measurements (or an output signal) based at least in part on a calibration for the infrared spectrum of a specific type of material (in this case, a person) that is being observed. This approach allows many infrared sensors to determined inferred temperatures accurately and remotely. However, without calibration to the type of material being observed, a passive infrared sensor may not be able to accurately determine the actual temperature values of, e.g., the group of individuals 114. In addition, even with the calibration, many infrared sensors may be sensitive to changes in the environmental condition, which may degrade the accuracy of the inferred temperatures of the group of individuals 114. This sensitivity to the environmental condition may be increased when the measurements are performed remotely, such as at distances greater than a few inches or centimeters from a given individual. By determining and using the parameter of the distribution of inferred temperatures, the monitoring techniques may be able to infer temperatures of the group of individuals 114 with or without a calibration for the infrared spectrum of a specific type of material that is being observed, in the presence of a change in the environmental condition, and/or when the measurements are performed remotely (such as at a distance greater than 1-6 ft).

In some embodiments, electronic device 112 may include one or more additional types of measurement sensors (ATMS) 134 that remotely perform one or more additional types of measurements on the group of individuals 114. Alternatively or additionally, electronic device 112 may acquire, via network 124, one or more additional measurements from one or more additional types of measurement sensors (not shown) in environment 110, such as an electronic scale that the group of individuals 114 may stand on as they approach portal 116. For example, the one or more additional types of measurements may include: acoustic measurements, optical imaging, LIDAR measurements, radar measurements (such as pulsed or FMCW radar measurements in which radar signals having a fundamental or carrier frequency in the radio frequency band may be transmitted, and reflected radar signals from an object may be received), and/or weight measurements. Note that the one or more additional types of measurements may be performed concurrently with the measurements. However, in other embodiments at least some of the measurements and the one or more additional types of measurements are performed sequentially, such as during the remedial action.

Furthermore, integrated circuit 120 may analyze the one or more additional types of measurements to determine: a type of vital sign of a given individual in the group of individuals 114, whether the given individual in the group of individuals 114 is sweating, a breathing style of the given individual in the group of individuals 114, a walking speed of the given individual in the group of individuals 114, a gait style of the given individual in the group of individuals 114, etc. For example, integrated circuit 120 may analyze the radar measurements to determine a range, an angle and/or a velocity (or Doppler information) of at least a portion of the given individual in the group of individuals 114. This may allow integrated circuit 120 to determine a pulse rate or condition (such as bradycardia or tachycardia) and/or a respiration rate or condition (such as bradypnea or tachypnea) of the given individual in the group of individuals 114. Alternatively or additionally, integrated circuit 120 may analyze the radar measurements or the LIDAR measurements to determine whether the given individual in the group of individuals 114 is breathing predominantly using their intercostal muscles or their diaphragm, has dyspnea (or difficulty breathing), has hyperpnea (or a large breathing volume), has kussmaul breathing (or a pattern of fast, deep breaths), etc., which may indicate or may be associated with a respiratory infection (such as a lung infection or pneumonia) or another disease state. Moreover, integrated circuit 120 may analyze the one or more additional types of measurements to determine whether the given individual in the group of individuals 114 is coughing, sneezing, has a runny nose, etc. Furthermore, integrated circuit 120 may analyze the one or more additional types of measurements to determine whether the given individual in the group of individuals 114 is walking briskly or slowly (relative to an average walking speed, e.g., of the group of individuals 114). In some embodiments, integrated circuit 120 may analyze the one or more additional types of measurements to determine the gait style of the given individual in the group of individuals 114, such as: a hemiplegic gait (e.g., with unilateral weakness), a diplegic gait (e.g., with bilateral lower extremity spasticity), a neuropathic gait (or a steppage gait or equine gait, in which there is a foot drop or weakness), a myopathic gait (with unilateral or bilateral pelvic weakness), a choreiform gait (or hyperkinetic gait, in which there are jerky movements), a ataxic gait (or cerebellar, in which there are clumsy staggering movements), a Parkinsonian gait (in which there is rigidity and slow movement with small step size), a sensory gait (or stomping gait, in which the feet are slammed down during walking), etc.

Note that while processing or analyzing the measurements and/or the one or more additional types of measurements, integrated circuit 120 may perform additional operations to extract or determine information. For example, integrated circuit 120 may perform windowing or filtering, one or more Fourier or discrete Fourier transforms (with at least 128 or 256 bits), peak detection, etc. In some embodiments, a constant false alarm rate (CFAR) technique is used to identify or determine whether a peak in received reflected radar signals is significant. Notably, integrated circuit 120 may calculate statistical metrics (such as a mean and a standard deviation) for a given range, and integrated circuit 120 may determine if a given radar peak is significant based on the calculated statistical metrics at different ranges. This approach may allow integrated circuit 120 to statistically identify or determine information associated with the given individual in the group of individuals 114.

Additionally, integrated circuit 120 may identify the individual based at least in part on the one or more additional types of measurements, such as the analysis of the one or more additional types of measurements. For example, an individual may be identified as being potentially unwell when they are determined to have one or more of: an inferred temperature that is an outliner in the distribution of inferred temperatures, an elevated pulse rate, an elevated respiration rate, a breathing style associated with a respiratory infection, coughing, sneezing, a runny nose, sweating, a slow walking speed, an abnormal gait, and/or another result of the one or more additional types of measurements. Thus, the one or more additional types of measurements may be used to improve the accuracy of the information and/or to provide additional information about the wellness of the individual.

In some embodiments, integrated circuit 120 may analyze the measurements and/or the one or more additional types of measurements using one or more machine-learning models and/or one or more neural networks (such as a machine-learning model and/or a neural network that is used with at least some of the measurements and/or the one or more additional types of measurements). For example, the machine-learning model and/or the neural network (such as a convolutional neural network) may be pretrained or predetermined to identify the individual based at least in part on the measurements and/or the one or more additional types of measurements. Notably, the machine-learning model and/or the neural network may be pretrained or predetermined using a dataset with individuals having characteristics (such as inferred temperatures, the one or more additional types of measurements, etc.) and associated labels (e.g., potentially unwell or well). Thus, the one or more machine-learning models and/or the one or more neural networks may perform classification on the group of individuals 114.

Note that inputs to the machine-learning model and/or the neural network may include extracted features in the measurements and/or the one or more additional types of measurements. For example, one or more optical images may be analyzed to extract features, including: edges associated with one or more potential objects in the one or more optical images, corners associated with the potential objects, lines associated with the potential objects, conic shapes associated with the potential objects, color regions within the optical image, and/or texture associated with the potential objects. In some embodiments, the features are extracted using a description technique, such as: scale invariant feature transform (SIFT), speed-up robust features (SURF), a binary descriptor (such as ORB), binary robust invariant scalable keypoints (BRISK), fast retinal keypoint (FREAK), etc. Moreover, the one or more machine-learning models may be trained or determined using one or more unsupervised or supervised machine-learning techniques, such as: a clustering technique, support vector machines, classification and regression trees, logistic regression, least absolute shrinkage and selection operator (LASSO), ridge regression, linear regression and/or another (linear or nonlinear) supervised-learning technique.

While the preceding discussion illustrated the use of the monitoring techniques to identify an individual that may have an infectious disease (such as a bacterial or a viral infection), in some embodiments the monitoring techniques may be used to identify an individual that is suspected of: having a different type of disease (including a non-infectious disease), being under the influence of a substance (such as alcohol, a prescription medication, an illegal drug, etc.), and/or having an altered mental state (e.g., due to a medical condition, substance abuse, mental illness, etc.). However, note that the monitoring techniques are intended for monitoring of environment 110, not for diagnosing what is wrong with the identified individual or performing a related medical assessment or intervention. Consequently, while electronic device 112 may identify that the individual is potentially unwell, this is not the same as determining or concluding that the individual is, in fact, sick. Instead, the monitoring techniques are used to provide efficient (e.g., rapid and low-cost) and remote assessment of the group of individuals 114 in order to identify an individual who is likely to be unwell, and then to selectively perform the remedial action in order to mitigate or reduce the potential harm to others that may occur if they are in proximity or contact with the identified individual.

Moreover, while the preceding discussion illustrated electronic device 112 with a particular number of components, in other embodiments electronic device 112 may include additional components, fewer components, two or more components may be combined into a single component, a component may be divided into two or more separate components, and/or a position of one or more components may be changed.

For example, there may be multiple radar transmitters and/or multiple radar receivers in or associated with electronic device 112. In some embodiments, the multiple transmitters in electronic device 112 may use beamforming to direct radar signals towards an object in environment 110. Additionally, a radar transmitter and a radar receiver in electronic device 112 may have separate transmit and receive antennas or may share an antenna between transmit and receive operations (e.g., duplex operation). Note that a transmit antenna and/or a receive antenna may include single or multiple radiators depending on the gain and beam width of a particular antenna configuration. In general, a transmit antenna and/or a receive antenna may have the same or different sizes. Moreover, a transmit antenna and/or a receive antenna may incorporate the same or different number and/or configurations of radiators. Furthermore, adjacent instances of a transmit antenna and/or a receive antenna may be separated by greater than or equal to one half of the average carrier frequency in a transmit radar signal.

Note that a transmit antenna and/or a receive antenna may be implemented using a wide variety of antenna structures and fabrication techniques, including an etched printed-circuit-board antenna or a multi-layer printed circuit board antenna with: microstrip feed lines and one or more patch radiators (which is sometimes referred to as a ‘patch antenna’), a substrate integrated waveguide (SIW) feed line and one or more SIW slotted radiator, coplanar waveguide feed lines with one or more SIW slotted radiator, a radiating stub antenna and/or other types of feed and radiator structures. In some embodiments, a transmit antenna and/or a receive antenna include high-gain antennas. In some embodiments, a transmit antenna has 6-30 dB gain, a beam width between 1° and 180° (such as a patch antenna having a beam width of 16 or 17°), a transmit power of up to 12 dBm, and an effective range up to 10-20 m.

Furthermore, while the preceding discussion illustrated the monitoring techniques in a single environment 110, in other embodiments the monitoring techniques may be implemented by multiple instances of electronic device 112 in multiple different embodiments. This approach may improve the statistics (and, thus, the accuracy) of the distribution of inferred temperatures by increasing the number of individuals in groups of individuals that are used to determine the distribution of inferred temperatures using measurements performed in the multiple environments. In addition, this approach may allow one or more hotspots to be determined (e.g., one or more environments with more identified individuals that are potentially unwell), which may allow variations in the prevalence of an infectious disease in a population or a region to be determined.

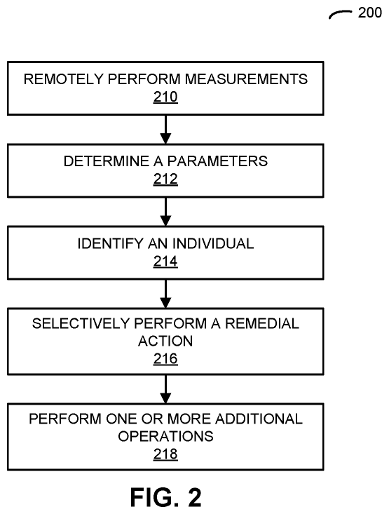

We now discuss embodiments of the method. FIG. 2 presents a flow diagram illustrating an example of a method 200 for selectively performing a remedial action. This method may be performed by an electronic device, such as electronic device 112 in FIG. 1.

During operation, the electronic device may remotely perform, using a measurement sensor, measurements (operation 210) corresponding to temperatures of a group of individuals. For example, the measurement sensor may include an infrared sensor, such as a thermopile or a set of thermocouples. Moreover, the measurement sensor may have an accuracy that is greater than medical grade, such as an inferred temperature accuracy greater than 0.1 or 0.5 C. Consequently, the measurement sensor may provide qualitative or relative measurements of the temperatures. Note that the remote measurements may be performed on the group of individuals at a distance greater than a fraction of an average height of the group of individuals.

Then, the electronic device may determine a parameter (operation 212) corresponding to a distribution of inferred temperatures of the group of individuals based at least in part on the measurements. For example, the parameter may include or may be related to a moment of the distribution, such as an average, a mean and/or a non-zero number times a standard deviation of the distribution. Alternatively or additionally, the parameter may include a percentile or may correspond to a portion of the distribution, such as a 75^{th}, 90^{th} or 95^{th} percentile. Note that the determining of the parameter corresponding to the distribution of the inferred temperatures may calibrate the measurement sensor for a change in an environmental condition, such as a change in a temperature of an environment that includes the group of individuals and the measurement sensor.

Moreover, the electronic device may identify an individual (operation 214) in the group of individuals based at least in part on the determined parameter and an inferred temperature of the individual. Note that the identified individual may be potentially unwell.

Next, the electronic device may selectively perform the remedial action (operation 216) based at least in part on the identification of the individual. For example, the remedial action may include: providing an acoustic or a visual alert; providing a message; performing one or more additional measurements on the individual; identifying one or more other individuals that have been in proximity with the individual during a time interval; enforcing a social distance between the individual and a remainder of the group of individuals; and/or changing an access control state of a portal.

In some embodiments, the electronic device optionally performs one or more additional operations (operation 218). Notably, the electronic device may remotely perform one or more additional types of measurements on the group of individuals using one or more additional types of measurement sensors. For example, the one or more additional types of measurements may include: acoustic measurements, optical imaging, LIDAR, radar measurements, and/or weight measurements. Note that the radar measurements may use an FMCW radar signal, where a carrier frequency of the FMCW radar signal has a predefined variation as a function of time (such as: a sine wave, a sawtooth wave or a chirp, a triangle wave and/or a square wave) over a predefined frequency range. Moreover, note that the one or more additional types of measurements may be performed concurrently with the measurements. However, in other embodiments at least some of the measurements and the one or more additional types of measurements are performed sequentially.

Furthermore, the integrated circuit may analyze the one or more additional types of measurements to determine: a type of vital sign of the group of individuals, breathing styles of the group of individuals, walking speeds of the group of individuals, gait styles of the group of individuals, etc. Additionally, the individual may be identified based at least in part on the one or more additional types of measurements, such as the analysis of the one or more additional types of measurements. In some embodiments, the measurements and/or the one or more additional types of measurements are analyzed using a machine-learning model and/or a neural network. For example, the machine-learning model and/or the neural network may be pretrained or predetermined to identify the individual based at least in part on the measurements and/or the one or more additional types of measurements.

In some embodiments of method 200 there may be additional or fewer operations. Moreover, the order of the operations may be changed, and/or two or more operations may be combined into a single operation.

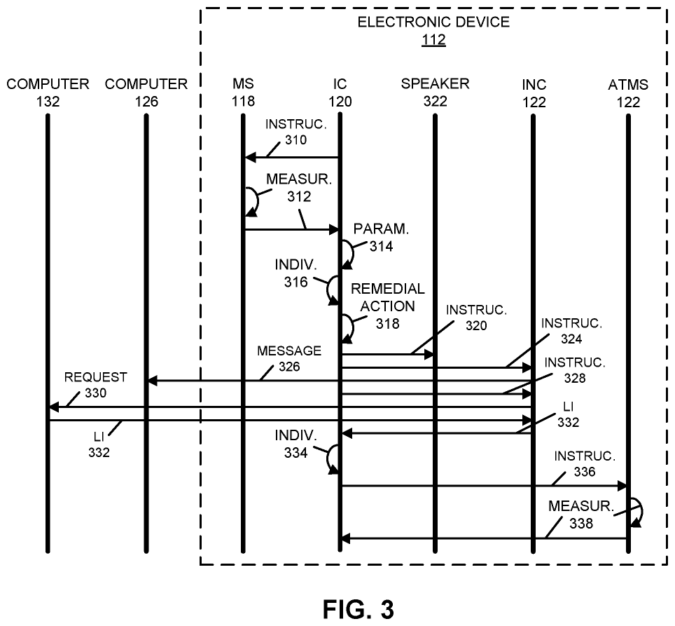

Embodiments of the monitoring techniques are further illustrated in FIG. 3, which presents a drawing illustrating an example of communication among components in electronic device 112. Notably, integrated circuit 120 (such as a processor) in electronic device 112 may provide an instruction 310 or a control signal to measurement sensor 118 in or associated with electronic device 112. In response, measurement sensor 118 may remotely (e.g., from a distance, such as more than 1-6 ft) perform measurements 312 corresponding to temperatures of a group of individuals. Then, measurement sensor 118 may provide measurements 312 and/or inferred temperatures to integrated circuit 120.

Moreover, integrated circuit 120 may determine a parameter 314 corresponding to a distribution of inferred temperatures of the group of individuals based at least in part on measurements 312. Furthermore, integrated circuit 120 may identify an individual 316 (or may provide information that specifies individual 316) in the group of individuals based at least in part on the determined parameter 314 and an inferred temperature of the individual. Note that the identified individual may be potentially unwell.

Next, integrated circuit 120 may selectively perform a remedial action 318 based at least in part on the identification of individual 316. For example, integrated circuit 120 may provide an instruction 320 or a control signal to a speaker 322 in or associated with electronic device 112 to output an alert. Alternatively or additionally, integrated circuit 120 may provide an instruction 324 to interface circuit 122 in electronic device 112. In response, interface circuit 122 may provide a message 326 to computer 126 with a notification about the identification of individual 316.

Moreover, integrated circuit 120 may provide an instruction 328 to interface circuit 122 to request 330, from computer 132, location information (LI) 332 of individuals. In response, computer 132 may provide location information 332 to electronic device 112. After receiving location information 332, interface circuit 122 may provide location information 332 to integrated circuit 310, which may use location information 332 to determine one or more individuals 334 (or information specifying the one or more individuals 334) who were or may have been in proximity to individual 316 during a time interval.

Furthermore, integrated circuit 120 may provide an instruction 336 or a control sensor to one or more additional types of measurement sensors 122 in electronic device. In response, the one or more additional types of measurement sensors 122 may remotely (e.g., from a distance, such as more than 1-6 ft) perform one or more measurements 338 associated with the individual and/or the group of individuals. Then, the one or more additional types of measurement sensors 122 may provide the one or more measurements 338 to integrated circuit 120, which may use the one or more measurements 338 to improve the accuracy of the identification of individual 316 and/or to determine whether to selectively perform remedial action 318.

While communication between the components in FIG. 3 is illustrated with unilateral or bilateral communication (e.g., lines having a single arrow or dual arrows), in general a given communication operation may be unilateral or bilateral.

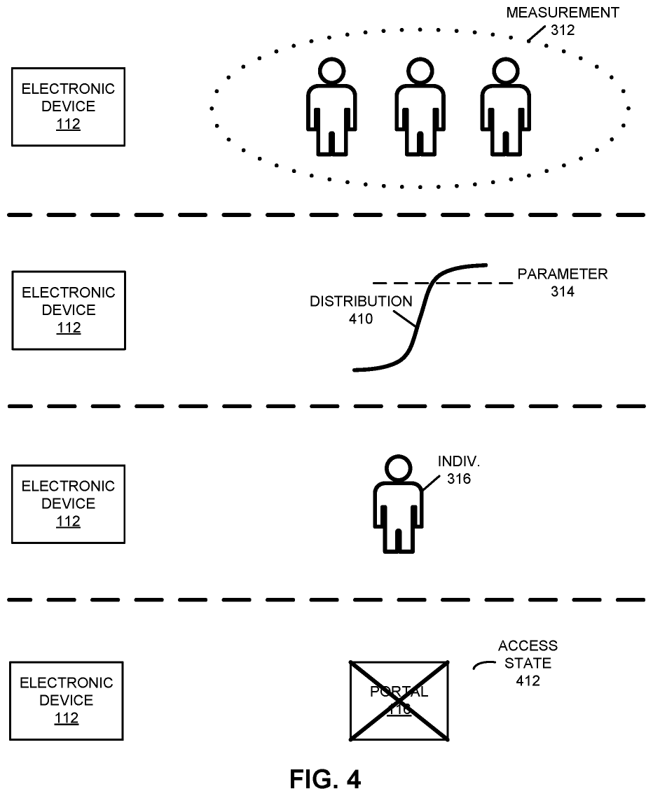

FIG. 4 presents a drawing illustrating an example of monitoring wellness using electronic device 112. Notably, electronic device 112 may remotely perform measurements 312 corresponding to temperatures of the group of individuals 114. Then, electronic device 112 may determine parameter 314 corresponding to a distribution 410 of inferred temperatures (such as a cumulative distribution) of the group of individuals 114 based at least in part on measurements 312. Moreover, electronic device 112 may identify individual 316 in the group of individuals 114 based at least in part on the determined parameter 314 and an inferred temperature of individual 320. For example, when the inferred temperature is in a 90^{th} percentile of the distribution of inferred temperatures 410, individual 316 may be identified as being potentially unwell. Next, electronic device 112 may selectively performing remedial action 318 based at least in part on the identification of individual 320, such as changing an access state 412 of portal 116.

In some embodiments, the individual may be identified based at least in part on the measurements using a sorting technique. Notably, electronic device 112 may perform multiple measurements on a given individual in the group of individuals 114 during a period of time (such as 5-10 s). Then, electronic device may sort these measurements from smallest to largest. A cumulative distribution of the sorted measurements may have an ‘S shape’. This cumulative distribution for the given individual in the group of individuals 114 may be compared to an average cumulative distribution for the group of individuals 114 (which may be determined by electronic device 112 based at least in part on multiple measurements performed for each of the individuals in the group of individuals 114). For example, the average cumulative distribution may be subtracted from the cumulative distribution of the given individual in the group of individuals 114.

Moreover, the difference between the cumulative distribution and the average cumulative distribution may be an indication of the wellness of the given individual in the group of individuals 114. For example, if the cumulative distribution and the average cumulative distribution are the same, the difference may be a horizontal line. In this case, the given individual in the group of individuals 114 may be deemed well (e.g., unlikely to be unwell). The larger the deviation of the difference from a horizontal line, the more likely that given individual in the group of individuals 114 may be potentially unwell (and, thus, identified by electronic device 112).

In some embodiments, a training dataset with well and unwell individuals is used to determine the parameter or a threshold for identifying the individual. For example, based at least in part on the training dataset, a machine-learning model or a neural network may be trained to identify the individual based at least in part on the shape of the difference between the cumulative distribution of a given individual in the training dataset and the average cumulative distribution of the training dataset (or the well individuals in the training dataset). In this way, the waveforms or shapes of the differences between the cumulative distributions of the unwell individuals in the training dataset and the average cumulative distribution of the training dataset (or the well individuals in the training dataset) may be determined, which may allow electronic device 112 to identify the individual that is potentially unwell based at least in part on the measurements.

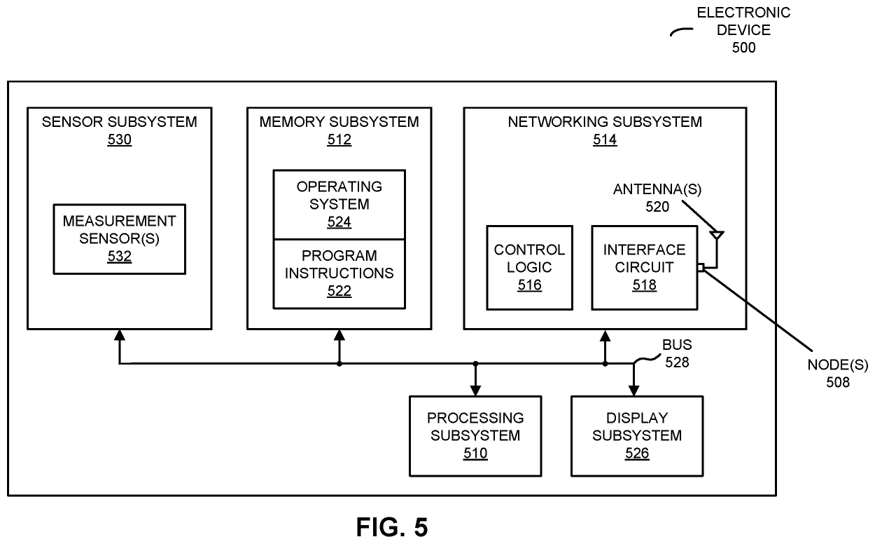

We now describe embodiments of an electronic device, which may perform at least some of the operations in the monitoring techniques. FIG. 5 presents a block diagram illustrating an example of an electronic device 500, such as electronic device 112 in FIG. 1. This electronic device may include processing subsystem 510, memory subsystem 512, networking subsystem 514 and sensor subsystem 530. Processing subsystem 510 includes one or more devices configured to perform computational operations. For example, processing subsystem 510 can include one or more microprocessors, ASICs, microcontrollers, programmable-logic devices, graphical processor units (GPUs) and/or one or more digital signal processors (DSPs).

Memory subsystem 512 includes one or more devices for storing data and/or instructions for processing subsystem 510 and networking subsystem 514. For example, memory subsystem 512 can include dynamic random access memory (DRAM), static random access memory (SRAM), and/or other types of memory (which collectively or individually are sometimes referred to as a ‘computer-readable storage medium’). In some embodiments, instructions for processing subsystem 510 in memory subsystem 512 include: one or more program modules or sets of instructions (such as program instructions 522 or operating system 524), which may be executed by processing subsystem 510. Note that the one or more computer programs may constitute a computer-program mechanism. Moreover, instructions in the various modules in memory subsystem 512 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 510.

In addition, memory subsystem 512 can include mechanisms for controlling access to the memory. In some embodiments, memory subsystem 512 includes a memory hierarchy that comprises one or more caches coupled to memory in electronic device 500. In some of these embodiments, one or more of the caches is located in processing subsystem 510.

In some embodiments, memory subsystem 512 is coupled to one or more high-capacity mass-storage devices (not shown). For example, memory subsystem 512 can be coupled to a magnetic or optical drive, a solid-state drive, or another type of mass-storage device. In these embodiments, memory subsystem 512 can be used by electronic device 500 as fast-access storage for often-used data, while the mass-storage device is used to store less frequently used data.

Networking subsystem 514 includes one or more devices configured to couple to and communicate on a wired and/or wireless network (e.g., to perform network operations), including: control logic 516, an interface circuit 518 and one or more antennas 520 (or antenna elements). (While FIG. 5 includes one or more antennas 520, in some embodiments electronic device 500 includes one or more nodes, such as nodes 508, e.g., a pad or a connector, which can be coupled to the one or more antennas 520 or a cable. Thus, electronic device 500 may or may not include the one or more antennas 520.) For example, networking subsystem 514 can include a Bluetooth networking system, a cellular networking system (e.g., a 3G/4G/5G network such as UMTS, LTE, etc.), a USB networking system, a networking system based on the standards described in IEEE 802.11 (e.g., a Wi-Fi networking system), an Ethernet networking system, and/or another networking system.

Note that a transmit or receive antenna pattern (or antenna radiation pattern) of electronic device 500 may be adapted or changed using pattern shapers (such as reflectors) in one or more antennas 520 (or antenna elements), which can be independently and selectively electrically coupled to ground to steer the transmit antenna pattern in different directions. (Alternatively or additionally, the transmit or receive antenna pattern may be adapted or changed using a phased array.) Thus, if one or more antennas 520 include N antenna pattern shapers, the one or more antennas may have 2^{N} different antenna pattern configurations. More generally, a given antenna pattern may include amplitudes and/or phases of signals that specify a direction of the main or primary lobe of the given antenna pattern, as well as so-called ‘exclusion regions’ or ‘exclusion zones’ (which are sometimes referred to as ‘notches’ or ‘nulls’). Note that an exclusion zone of the given antenna pattern includes a low-intensity region of the given antenna pattern. While the intensity is not necessarily zero in the exclusion zone, it may be below a threshold, such as 3 dB or lower than the peak gain of the given antenna pattern. Thus, the given antenna pattern may include a local maximum (e.g., a primary beam) that directs gain in the direction of electronic device 500 that is of interest, and one or more local minima that reduce gain in the direction of other electronic devices that are not of interest. In this way, the given antenna pattern may be selected, e.g., to target an object of interest in an environment of electronic device 500.

Networking subsystem 514 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between the electronic devices does not yet exist. Therefore, electronic device 500 may use the mechanisms in networking subsystem 514 for performing simple wireless communication between the electronic devices, e.g., transmitting frames and/or scanning for frames transmitted by other electronic devices.

Within electronic device 500, processing subsystem 510, memory subsystem 512, and networking subsystem 514 are coupled together using bus 528. Bus 528 may include an electrical, optical, and/or electro-optical connection that the subsystems can use to communicate commands and data among one another. Although only one bus 528 is shown for clarity, different embodiments can include a different number or configuration of electrical, optical, and/or electro-optical connections among the subsystems.

In some embodiments, electronic device 500 includes an optional display subsystem 526 for displaying information on a display, which may include a display driver and the display, such as a liquid-crystal display, a multi-touch touchscreen, etc.

Furthermore, electronic device 500 may include a sensor subsystem 530, which may include one or more measurement sensors 532. In some embodiments, sensor subsystem 530 includes one or more: a temperature sensor (such as an infrared sensor), a radar sensor, an acoustic sensor, an imaging sensor, LIDAR, etc. These measurement sensors may be used separately or in conjunction with each other.

Electronic device 500 can be (or can be included in) a wide variety of electronic devices. For example, electronic device 500 can be (or can be included in): a desktop computer, a laptop computer, a subnotebook/netbook, a server, a computer, a mainframe computer, a cloud-based computer, a tablet computer, a smartphone, a cellular telephone, a smartwatch, a consumer-electronic device, a portable computing device, a transceiver, a measurement device, another electronic device and/or a vehicle.

Although specific components are used to describe electronic device 500, in alternative embodiments, different components and/or subsystems may be present in electronic device 500. For example, electronic device 500 may include one or more additional processing subsystems, memory subsystems, networking subsystems, display subsystems and/or sensor subsystems. Additionally, one or more of the subsystems may not be present in electronic device 500. Moreover, in some embodiments, electronic device 500 may include one or more additional subsystems that are not shown in FIG. 5. Also, although separate subsystems are shown in FIG. 5, in some embodiments some or all of a given subsystem or component can be integrated into one or more of the other subsystems or component(s) in electronic device 500. For example, in some embodiments program instructions 522 are included in operating system 524 and/or control logic 516 is included in interface circuit 518.

Moreover, the circuits and components in electronic device 500 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit (which is sometimes referred to as a ‘communication circuit’ or a ‘means for communication’) may implement some or all of the functionality of networking subsystem 514 or sensor subsystem 530. The integrated circuit may include hardware and/or software mechanisms that are used for transmitting wireless or radar signals from electronic device 500 and receiving wireless or radar signals at electronic device 500 from other electronic devices. Aside from the mechanisms herein described, radios are generally known in the art and hence are not described in detail. In general, networking subsystem 514 and/or the integrated circuit can include any number of radios. Note that the radios in multiple-radio embodiments function in a similar way to the described single-radio embodiments.

In some embodiments, networking subsystem 514 and/or the integrated circuit include a configuration mechanism (such as one or more hardware and/or software mechanisms) that configures the radio(s) to transmit and/or receive on a given communication channel (e.g., a given carrier frequency). For example, in some embodiments, the configuration mechanism can be used to switch the radio from monitoring and/or transmitting on a given communication channel to monitoring and/or transmitting on a different communication channel. (Note that ‘monitoring’ as used herein comprises receiving signals from other electronic devices and possibly performing one or more processing operations on the received signals)

Moreover, another integrated circuit may implement some or all of the functionality related to the monitoring techniques.

In some embodiments, an output of a process for designing a given integrated circuit, or a portion of the given integrated circuit, which includes one or more of the circuits described herein may be a computer-readable medium such as, for example, a magnetic tape or an optical or magnetic disk. The computer-readable medium may be encoded with data structures or other information describing circuitry that may be physically instantiated as the given integrated circuit or the portion of the given integrated circuit. Although various formats may be used for such encoding, these data structures are commonly written in: Caltech Intermediate Format (CIF), Calma GDS II Stream Format (GDSII) or Electronic Design Interchange Format (EDIF). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the monitoring techniques may be implemented using program instructions 522, operating system 524 (such as a driver for interface circuit 518) or in firmware in interface circuit 518. Alternatively or additionally, at least some of the operations in the monitoring techniques may be implemented in a physical layer, such as hardware in interface circuit 518 or sensor subsystem 530.

In the preceding description, we refer to ‘some embodiments.’ Note that ‘some embodiments’ describes a subset of all of the possible embodiments, but does not always specify the same subset of embodiments. Moreover, numerical values in the preceding embodiments are illustrative examples of some embodiments. In other embodiments of the monitoring techniques, different numerical values may be used.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. An electronic device, comprising:
a measurement sensor, having a field of view, configured to remotely perform measurements corresponding to temperatures of a group of individuals; and
an integrated circuit, coupled to the measurement sensor, configured to analyze the measurements, wherein the electronic device is configured to:
remotely perform, using the measurement sensor, measurements corresponding to the temperatures of a group of individuals;
determine, using the integrated circuit, a parameter corresponding to a distribution of inferred temperatures of the group of individuals based at least in part on the measurements;
identify, using the integrated circuit, an individual in the group of individuals based at least in part on the determined parameter and an inferred temperature of the individual; and
selectively perform a remedial action based at least in part on the identification of the individual.

2. The electronic device of claim 1, wherein the measurement sensor comprises an infrared sensor.

3. The electronic device of claim 1, wherein the measurement sensor has an accuracy that is greater than 0.5 C.

4. The electronic device of claim 1, wherein the measurement sensor is configured to provide qualitative or relative measurements of the temperatures.

5. The electronic device of claim 1, wherein the parameter comprises: a moment of the distribution, corresponds to the moment of the distribution, or a percentile or a portion of the distribution.

6. The electronic device of claim 1, wherein the identified individual is potentially unwell.

7. The electronic device of claim 1, wherein the remedial action comprises: providing an alert; providing a message; performing one or more additional measurements on the individual; identifying one or more other individuals that have been in proximity with the individual during a time interval; enforcing a social distance between the individual and a remainder of the group of individuals; or changing an access control state of a portal.

8. The electronic device of claim 1, wherein the determining of the parameter corresponding to the distribution of the inferred temperatures calibrates the measurement sensor for a change in an environmental condition.

9. The electronic device of claim 1, wherein measurement sensor is configured to perform the measurements on the group of individuals at a distance greater than a fraction of an average height of the group of individuals.

10. The electronic device of claim 1, wherein the electronic device comprises one or more additional types of measurement sensors configured to remotely perform one or more additional types of measurements on the group of individuals; and
wherein the individual is identified based at least in part on the one or more additional types of measurements.

11. The electronic device of claim 10, wherein the one or more additional types of measurements comprise: acoustic measurements, optical imaging, LIDAR, radar measurements, or weight measurements.

12. The electronic device of claim 10, wherein the radar measurements use a frequency-modulated continuous-wave (FMCW) radar signal; and
wherein a carrier frequency of the FMCW radar signal has a predefined variation as a function of time over a predefined frequency range.

13. The electronic device of claim 10, wherein the one or more additional types of measurements are performed concurrently with the measurements.

14. The electronic device of claim 10, wherein at least some of the measurements and the one or more additional types of measurements are performed sequentially.

15. The electronic device of claim 10, wherein the electronic device is configured to analyze, using the integrated circuit, the one or more additional types of measurements to determine: a type of vital sign of the group of individuals, breathing styles of the group of individuals, walking speeds of the group of individuals, or gait styles of the group of individuals.

16. The electronic device of claim 10, wherein the electronic device is configured to analyze the measurements, the one or more additional types of measurements or both using a machine-learning model or a neural network.

17. A non-transitory computer-readable storage medium for use in conjunction with an electronic device, the computer-readable storage medium configured to store program instructions that, when executed by the electronic device, cause the electronic device to perform operations comprising:
remotely performing, using a measurement sensor, measurements corresponding to temperatures of a group of individuals;
determining a parameter corresponding to a distribution of inferred temperatures of the group of individuals based at least in part on the measurements;
identifying an individual in the group of individuals based at least in part on the determined parameter and an inferred temperature of the individual; and
selectively performing a remedial action based at least in part on the identification of the individual.

18. The non-transitory computer-readable storage medium of claim 17, wherein the measurement sensor provides qualitative or relative measurements of the temperatures.

19. The non-transitory computer-readable storage medium of claim 17, wherein the determining of the parameter corresponding to the distribution of the inferred temperatures calibrates the measurement sensor for a change in an environmental condition.

20. A method for selectively performing a remedial action, comprising:
by an electronic device:
remotely performing, using a measurement sensor, measurements corresponding to temperatures of a group of individuals;
determining a parameter corresponding to a distribution of inferred temperatures of the group of individuals based at least in part on the measurements;
identifying an individual in the group of individuals based at least in part on the determined parameter and an inferred temperature of the individual; and
selectively performing the remedial action based at least in part on the identification of the individual.
